(19) United States
(12) Patent Application Publication
(10) Pub. No.: US 2011/0015488 A1
(43) Pub. Date: Jan. 20, 2011

(54) Working conduit

(76) Inventor: Wei-Chen Hong, Tainan (TW)

Correspondence Address: BACON & THOMAS, PLLC, 625 SLATERS LANE, FOURTH FLOOR, ALEXANDRIA, VA, 22314-1176, US

(21) Appl. No.: 12/591,228

(22) Filed: Nov. 13, 2009

(30) Foreign Application Priority Data
Jul. 20, 2009 (TW) 098124361

(51) Int. Cl. A61B 1/06

(52) U.S. Cl. 600/176

(57) ABSTRACT

A working conduit comprises an inner tube and an outer tube into which the inner tube can be inserted. The inner tube is a transparent hollow tube of which one end is a close end, and the other end is an open end. The outer tube is also a transparent hollow tube of which both ends are open ends. The close end of the inner tube is configured as a semi-spherical shape, a curved surface, a streamline shape, a parabolic surface or a cone with a rounded tip. The inner tube can be used as an observation tube. An endoscope can be inserted into the inner tube for confirming the position, range, orientation and/or depth of the portion to be surgically treated.

### BACKGROUND OF THE INVENTION

1. Field of the invention

The present invention relates to a working conduit, especially to a working conduit used in endoscopic surgery.

2. Brief Description of the Prior Art

Taiwanese Patent No. M 283609 entitled “Endoscope Working Conduit Used inside Skull” is disclosed in which a inner solid tube (i.e. a plug) and a hollow outer tube are included therein. In order to remove intracranial hematoma by endoscopic surgery, the wording conduit for endoscopic surgery has to be temporarily implanted or inserted into the brain organism. In turn, the inner tube is drawn out so that the outer tube remains and serves as the working conduit for endoscope.

### SUMMARY OF THE INVENTION

The present invention proposes a working conduit comprising a transparent hollow inner tube and a transparent hollow outer tube into which the inn tube is to be inserted. One end of the inner tube is a close end, and the other end of the inner tube is an open end. Both ends of the outer tube are open ends.

The close end of the inner tube is configured as a semi-spherical shape, a curved surface, a streamline shape, a parabolic surface or a cone with a rounded tip. The inner tube can be used as an observation tube, into which an endoscope can be inserted for confirming the position, range, orientation and/or depth of the portion to be surgically treated.

Preferably, the close end of the inner tube is configured as an optical element capable of providing the endoscope with a wide-angle view.

Preferably, the curvature of the outer wall surface of the close end of the inner tube is smaller than that of the inner wall surface of the close end of the inner tube.

Preferably, the wall thickness of the close end of the inner tube is increased inwardly radially.

### DETAILED DESCRIPTION OF PREFERRED EMBODIMENT

The technical features of the present invention will become more apparent from the detailed description of the preferred embodiment in conjunction with the accompanying drawings. The preferred embodiment is purely for descriptive purpose, not for limitation on the scope of the present invention. The accompanying drawings are also for illustrative purpose, which may not be drawn to scale.

FIG. 1 is an embodiment showing the working conduit according to the present invention, in which the whole working conduit is designated by 10. The working conduit 10 comprises an inner tube 12 and an outer tube 14.

The inner tube 12 and the outer tube 14 are formed by transparent material, such as glass, plastics or resin. As shown in FIG. 2, the inner tube 12 is formed into a hollow tube, which has a close end 121 and an open end 122. As shown in FIG. 3, the outer tube 14 is formed into a hollow tube, which has two open ends 141, 142. The outer diameter of the inner tube 12 is approximately equal to the inner diameter of the outer tube 14 such that the inner tube 12 can be inserted into the outer tube 14 with the close end 121 being exposed and extended from the open end 142 of the outer tube 14.

Optionally, a flange or a tab portion may be provided on the open end 122 of the inner tube 12 for the purpose of gripping, operating or positioning.

Optionally, a flange or a tab portion may be provided on the open end 142 of the outer tube 14 for the purpose of gripping, operating or positioning, and the lower open end 141 of the outer tube 14 may be chamfered so as to reduce friction during insertion and to avoid injuring the organism.

In order to reduce friction during insertion and to avoid injuring the organism, the close end 121 of the inner tube 12 is preferably configured as a semi-spherical surface, a curved surface, a streamline shape, a parabolic surface or a cone with a rounded tip.

The inner tube 12 of the working conduit according to the present invention may serve alone as an observation tube into which an endoscope 42 is inserted for confirming the position, orientation and/or depth of the portion to be surgically treated. Thus, the close end 121 of the inner tube 12 is advantageously configured as an optical element capable of providing the endoscope 42 inserted in the inner tube 12 with a wide-angle view.

For example, the close end 121 of the inner tube 12 can be configured as a structure with a meniscus lens or a positive meniscus lens. Alternatively, the close end 121 of the inner tube 12 can be so configured that the curvature of the outer wall surface is smaller than that of the inner wall surface of the close end 121. Alternatively, the close end 121 of the inner tube 12 can be so configured that the wall thickness of the close end 121 is increased inwardly radially.

Then, the application of the working conduit 10 according to the present invention will be described in conjunction with a example of the endoscopic surgery for removing intracranial hematoma.

Intracranial hematoma is often occurred the intracranial part of a patient due to an apoplectic stroke or other reasons. Such hematoma should be removed because the hematoma may be a threat to the life of the patient.

After the diagnosis is made by the doctor, part of the scalp and part of the skull at the position corresponding to the hematoma are removed by surgery so as to expose the brain organism.

Before the working conduit is temporarily implanted into the brain organism, the inner tube 12 serving as the observation tube with an endoscope inserted therein is inserted into the exposed brain organism 50, as shown in FIG. 4. The doctor observes and furthermore confirms the position, range, orientation and/or depth of the hematoma 51 occurred through the endoscope 42.

Once the position, range, orientation and/or depth of the hematoma 51 is confirmed, the inner tube 12 serving as the observation tube is drawn out. In turn, as shown in FIG. 5, the inner tube 12 and the outer tube 14 are inserted into the brain organism 50 with the inner tube 12 being inserted into the outer tube 14. Finally, the inner tube 12 is drawn out, and the outer tube 14 remains and serves as a working conduit for endoscopic surgery, as shown in FIG. 6.

FIG. 7 is a schematic view showing that the outer tube 14 is used as an working conduit for endoscopic surgery. The endoscope 42 and a sucking means 43 penetrate through the outer tube 14 and reach the place of the hematoma 51 for removing the hematoma 51. Other tools such as hemostatic tool or hemostat may be used optionally.

While this invention has been described with reference to the embodiment, it should be understood that various changes and modifications could be made within the spirit and scope of the inventive concepts described. Accordingly, it is intended that the invention shall not be limited to the disclosed embodiment but have the full scope permitted by the language of the following claims.

### BRIEF DESCRIPTION OF THE DRAWINGS

FIG. 1 is an embodiment showing working conduit according to the present invention;

FIG. 2 is a view showing the inner tube of the working conduit;

FIG. 3 is a view showing the outer tube of the working conduit;

FIGS. 4 to 6 show the process for temporarily implanting the working conduit; and

FIG. 7 is a schematic view showing that the endoscopic surgery is performed through the working conduit of the present invention.

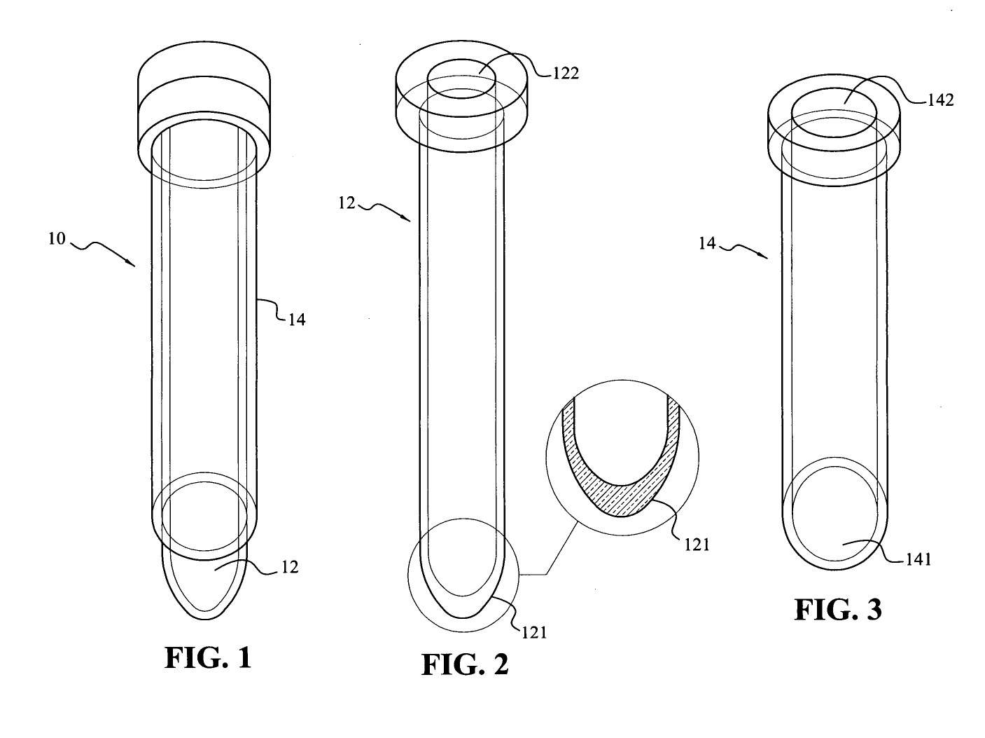

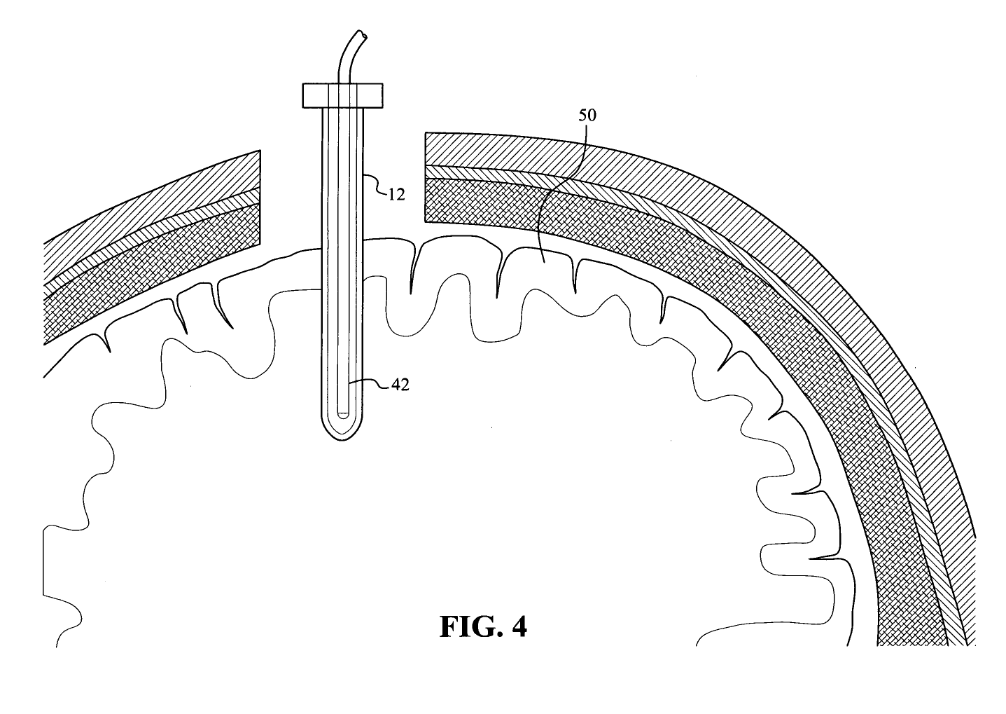

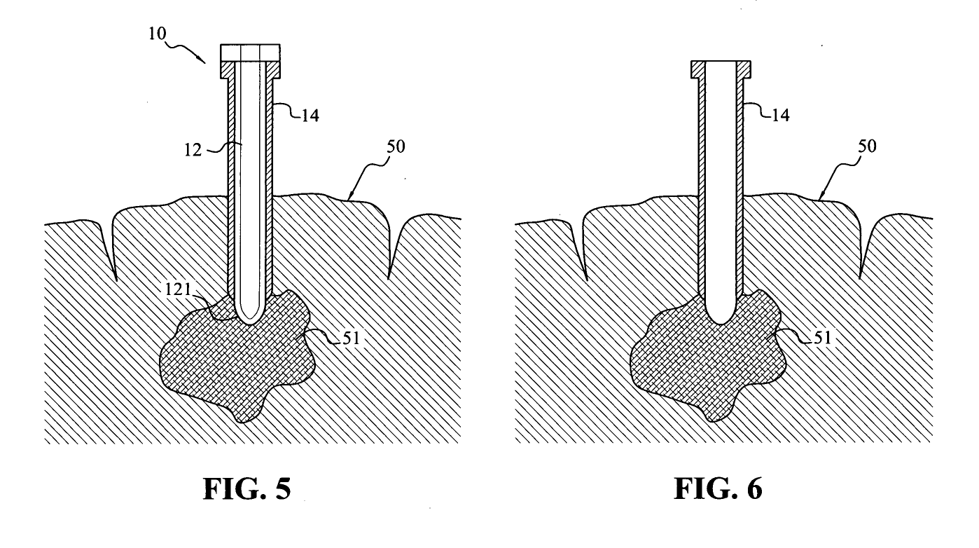

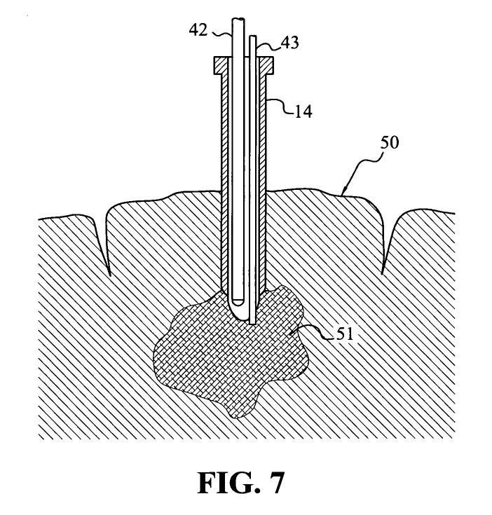

What is claimed is:

1. A working conduit, comprising an inner tube and an outer tube in which said inner tube is to be inserted into said outer tube, wherein:
said inner tube is a transparent hollow tube of which one end is a close end, and the other end is an open end;
said outer tube is a transparent hollow tube of which both ends are open ends;
said close end of the inner tube is configured as a semi-spherical shape, a curved surface, a streamline shape, a parabolic surface, or a cone with a rounded tip, and said inner tube is to be used as an observation tube, an endoscope being inserted into said inner tube to confirm a position, a range, an orientation and/or a depth of a portion to be conducted with surgery.

2. The working conduit as claimed in claim 1, wherein said close end of the inner tube is configured as an optical element capable of providing the endoscope with a wide-angle view.

3. The working conduit as claimed in claim 1, wherein said close end of the inner tube is configures as a structure with a meniscus lens.

4. The working conduit as claimed in claim 1, wherein said close end of the inner tube is configured as a structure with a positive meniscus lens.

5. The working conduit as claimed in claim 1, wherein a curvature of an outer wall surface of the closed end of the inner tube is smaller than that of an inner wall surface of the close end of the inner tube.

6. The working conduit as claimed in claim 1, wherein a wall thickness of said close end of the inner tube is incrementally increased inwardly radially.

7. The working conduit as claimed in claim 1, wherein one end of said outer tube and/or the open end of said inner tube are provided with a flange or a tab portion.

8. The working conduit as claimed in claim 1, wherein one end of said outer tube is chamfered.
